(19) United States
(12) Patent Application Publication
(10) Pub. No.: US 2022/0017852 A1
(43) Pub. Date: Jan. 20, 2022

(54) PROCESS AND APPARATUS FOR PRODUCING EXOSOMES

(71) Applicant: SCUOLA UNIVERSITARIA PROFESSIONALE DELLA SVIZZERA ITALIANA (SUPSI), Manno (CH)

(72) Inventors: Agatino Christian Tavilla, Agno (CH); Roberto Robortella, Odogno (CH); Daniele Nicolis, Lopagno (CH); Igor Sergio Laerte Stefanini, Cadro (CH); Lucio Barile, Canobbio (CH); Alessandro Favalli, Cremenaga (IT)

(21) Appl. No.: 17/299,643

(22) PCT Filed: Dec. 19, 2019
(86) PCT No.: PCT/IB2019/061074

§ 371 Date: Jun. 3, 2021

(30) Foreign Application Priority Data
Dec. 27, 2018 (IT) 102018000021226

(51) Int. Cl. C12M 1/00

(52) U.S. Cl. CPC C12M 47/02; C12M 29/18; C12M 47/10

(57) ABSTRACT

Process and apparatus for producing exosomes operating continuously, in which a product containing exosomes is extracted from an incubator (1); said product is subjected to tangential filtering in a concentrator (3); the product is fed in two separate flows (20, 23) to two sides (301, 302) of the concentrator (3) separated by a semi-permeable wall obtaining a concentration of the exosomes in one of the two flows; a flow of product enriched with exosomes (24) is extracted from the concentrator; a diluted product (30) after transit through the concentrator is recirculated to the incubator; thermal conditioning (2), gaseous conditioning (4) and in-line pH control are provided.

### FIELD OF APPLICATION

The present invention relates to a process and relative apparatus for producing exosomes.

### PRIOR ART

The exosomes as it is known are extracellular vesicles with a diameter generally from 30 nm to 150 nm, involved in the communication processes between cells.

There is considerable interest in exosomes in the therapeutic field, in which exosomes have proved to have useful applications. Consequently, there is a need for effective methods of producing exosomes intended for such use.

One of the reasons of interest is that the exosome therapy is not subject to the strict limitations of use of the cell therapy because exosomes are not cells, rather they are by-products of the cells themselves. Therefore the exosome therapy has a particularly wide potential of application.

The production of exosomes in the prior art is essentially based on an incubation culture, in which cells release exosomes into a culture medium. The incubation process therefore produces a material (conditioned medium) which essentially consists of an aqueous medium containing the exosomes in the form of a particulate.

The separation of the exosomes from the culture medium is carried out in the prior art with an intermittent batch type process by taking an amount of said material containing exosomes and subjecting it to centrifugation and ultra-centrifugation cycles.

This technique has the disadvantage of being able to process limited amounts of the material containing exosomes for each centrifugation cycle (typically a few milliliters at a time); given its nature as a batch process, it is difficult to scale to a greater production capacity. Another disadvantage of the batch process is that sampling and centrifugation require handling the material containing exosomes and therefore pose a high risk of contamination. Another problem is represented by the maintenance of the correct environmental conditions in the incubator, which is subjected to fluctuations for example in the case of integration of fresh medium from the outside.

To overcome the problems related to centrifugation, processes have been proposed which provide for filtration as suggested for example in the patent documents U.S. Pat. No. 6,899,863, US 2014/0099649 and US 2017/0029802. However, even these systems are not satisfactory for obtaining a large-scale production of exosomes, with low costs and with a reduced risk of contamination from the outside.

A cross-flow type process for isolating exosomes from a fluid, using a conventional centrifugation process, is described in McNamara et al. “Large-scale, cross-flow based isolation of highly pure and endocytosis-competent extracellular vesicles”, Journal of extracellular vesicles, vol. 7 No. 1.

### SUMMARY OF THE INVENTION

The invention aims to overcome the limits of the known art. The invention has the scope of a process of production of exosomes which can operate continuously with culture of cells that produce exosomes (cell culture with release of exosomes) and with in-line extraction of exosome. Another scope is to operate in a closed circuit kept sterile and with continuous control of environmental conditions including temperature, oxygen (O₂) and carbon dioxide (CO₂) concentration, pH. Another aim is to provide an apparatus for producing exosomes operating with a continuous process, with a highly automated operation, and with minimal operator intervention. A further aim is to provide a process and apparatus which allow to significantly increase the area of the cell culture with the same cost and size. Another aim is to provide a process and apparatus that are easily scalable in terms of exosome production capacity. Still another aim is that the process and apparatus are suitable for a serum-free culture where required.

The above aims are achieved with a process and an apparatus according to the claims.

A process according to the invention provides for the continuous production of exosomes through the steps of:

carrying out a cell culture with release of exosomes in an incubator obtaining a product containing exosomes and an aqueous culture medium,

continuously feeding a first portion of said product to a first side of a concentrator device,

continuously feeding a second portion of said product to a second side of said concentrator device,

wherein said first side and said second side of the concentrator device are in communication with each other via a semi-permeable wall which is permeable to the culture medium and substantially impermeable to the exosomes,

wherein the transit of a first flow and a second flow of said product through the first side and the second side of the concentrator device, respectively, causes a passage of the culture medium from the second side towards the first side of the concentrator, across said semi-permeable wall, and a consequent increase in the concentration of exosomes in the product which transits through the second side,

collecting a product enriched with exosomes from said second side of the concentrator device,

collecting a diluted product from said first side of the concentrator device and recirculating said diluted product to said incubator,

wherein the aforesaid steps are carried out in a closed loop, said closed loop comprising both the cell culture and the concentration of the exosomes released by said cell culture.

The process preferably comprising the further steps of: sending the product enriched with exosomes, which is extracted from the second side of the concentrator, to a collector device; extracting from the collector a concentrate of exosomes. Said exosome concentrate can be sent to one or more containers connected to the collector.

Preferably a part of the product enriched with exosomes sent to said collector is recirculated from the collector towards said second side of the concentrator. More preferably, also the steps of sending the product enriched with exosomes from the concentrator to the collector and recirculating said product from the collector to the concentrator are carried out in a closed loop.

The possibility of extracting one or more samples of exosomes through the collector without putting the closed circuit comprising the cell culture and the concentration of exosomes in communication with the outside represents an important advantage.

Advantageously, the product which is fed into the second side of the concentrator is at a higher pressure than the product fed into the first side. In this way, the passage of the culture medium from the second side to the first side of the concentrator is substantially unidirectional.

The product extracted from the incubator is a substantially liquid and pumpable product, which comprises an aqueous culture medium in which the particulate of exosomes is found. The term enriched product or diluted product indicates a product having respectively a concentration of exosomes greater than or less than the product extracted from the incubator.

The process advantageously envisages extracting a portion of waste product and reintegrating with fresh medium. These operations are performed in-line, preferably in the branch which feeds the first part of product to the first side of the concentrator, and before entry in the concentrator itself.

The process also provides for a constant and in-line control of the product conditions, particularly of the temperature, the concentration of oxygen and carbon dioxide and the pH.

Preferably the product directed to the first side of the concentrator is subjected to a thermal conditioning so that it enters the concentrator at a controlled temperature. The thermal conditioning can comprise heating or cooling and for example it compensates for the variation of temperature due to the introduction of fresh medium.

The diluted product extracted from the first side of the concentrator, and which is directed to a recirculation in the incubator, is preferably subjected to a conditioning step (called gas conditioning) which includes adding oxygen and/or carbon dioxide to the product itself to keep desired oxygenation and pH levels in the incubator and maximize the release of exosomes.

A sample of the product recirculated to the incubator can be taken and subjected to pH analysis to consequently control the described gas conditioning step. Said pH analysis step may require the addition of a reagent (called colorimetric reagent) to allow the detection of pH by a colorimetric sensor. In a preferred embodiment, the analysis is performed off-line so that the circuit between incubator and concentrator is not contaminated with said colorimetric reagent. The sample analysed as to the pH measurement is subsequently eliminated as a waste material in a suitable tank. In this way the process is compatible with clinical grade culture requirements.

The concentrator essentially performs a tangential filtering by separating the aqueous culture medium from the exosomes. The exosomes can be collected in the form of a solution with a higher concentration of exosomes.

The geometry of the first and second side of the concentrator is not essential for the purposes of the invention and can vary according to various embodiments. For example, the first side and the second side can comprise respectively a first chamber and a second chamber arranged side by side or arranged coaxially one around the other.

In a preferred embodiment, the concentrator comprises a rectangular cartridge system for tangential filtration.

In another embodiment, the concentrator is made as an essentially tubular device in which the two sides are represented by a first tubular chamber and a second cylindrical annular chamber which is arranged coaxially around the first chamber.

Preferably the two chambers of the concentrator are fed in a counter-current manner (flow opposition).

The semi-permeable wall preferably performs a dimensional filtering in a range between 100 kDalton and 300 kDalton.

A preferred material for the semi-permeable wall is polyethersulfone.

The product enriched with exosomes extracted from the second side of the concentrator is advantageously pumped to the aforementioned collector device and, from the latter, in suitable containers. Part of the enriched product can be recirculated from said collector to the second side of the concentrator.

An apparatus according to the invention (bio-reactor) can be advantageously made in the form of an essentially closed box with a basic circuit comprising the incubator, the concentrator, the collector, the thermal conditioner and the gas conditioner. Said basic circuit operates continuously and is a closed and sterile circuit that does not require user access during normal operation.

The handling of the various process fluids takes place with suitable devices such as pumps, valves and selectors which are known per se. The valves can comprise unidirectional valves and advantageously said unidirectional valves are actuated by the flows.

The points of contact of said closed circuit with the external environment may comprise the points of sampling of concentrate of exosomes and of connection to the conditioning gas. Preferably said points of contact are the only points of contact of the closed circuit with the external environment. These points of contact can be controlled by valves combined with suitable filters adapted to keep the main circuit (incubation and filtering) substantially isolated from the outside world in order to reduce the risk of contamination. Said filters are preferably 0.22 μm (micron) filters.

It should be noted that the incubator operates in a closed line without direct exchange of mass with the outside, since the gas conditioning (oxygenation and/or introduction of CO₂) takes place in line.

The process and the apparatus according to the invention allow performing in closed line: the cell culture; the reintegration of fresh medium; the thermal and gaseous reconditioning (O₂ and CO₂); the recirculation of product to and from the incubator; the concentration of exosomes. The filtering of the exosomes is also carried out continuously avoiding collection and centrifugation of the product and consequently reducing the risk of contamination considerably. This continuous filtering has the advantage of constantly reducing the concentration of exosomes in the cell culture thus inducing the cells in culture to produce a greater quantity of exosomes.

In-line thermal reconditioning is particularly advantageous when introducing refrigerated fresh medium as it avoids thermally buffering the medium in the incubator. The system according to the invention is able to automatically and constantly maintain the desired environmental conditions inside the main circuit and above all inside the incubator.

Advantages of the invention are essentially as follows: the incubation and filtering process of the exosomes is performed continuously and in a closed and sterile circuit; constantly controlled environment with regard to temperature, oxygenation and pH; reintegration of fresh medium and in-line sampling of exosomes; feasibility in the form of a closed device with automated fluid movement; minimum operator intervention required; scalability to high exosome production capacities; applicability at industrial scale.

In particular, a feature of the invention consists in the fact that the culture of the cells that release exosomes is integrated in a closed loop with the concentration of the exosomes in the form of culture medium enriched by these extracellular vesicles of endocytotic origin. The cell culture takes place in a special incubator which is in a closed loop comprising the devices for the concentration of the product (solution) containing exosomes and the extraction of a concentrate of exosomes.

The integration of the culture of exosomes-releasing cells with the concentration and separation process is advantageous compared to the processes of the known art which treat a source fluid (for example supernatant, plasma, tumour fluid) generated externally with respect to the concentration process. The generation of the source fluid outside the concentration process forces to manipulate the fluid with risk of contamination. The integration of the culture in the closed loop with the concentration steps eliminates this risk and gives a better assurance of sterility and an improved process control. The continuous extraction of exosomes from the cell culture, carried out over several weeks, also allows an interaction with the same cell culture optimizing both the growth curve of the same cell culture and the concentrated exosome material.

It should be noted that the treatment of the product extracted from the incubator advantageously does not include any centrifugation and ultra-centrifugation step.

In a particularly preferred application the exosomes produced are exosomes from cardiac cells or other mesenchymal cells that grow by adhesion.

Further characteristics and advantages will become more evident from the description that follows of a preferred embodiment, which is given as a non-limiting example.

### DESCRIPTION OF THE FIGURES

FIG. 1 shows the scheme of an apparatus according to an embodiment of the invention.

FIG. 2 is a scheme of concentrator usable in the apparatus of FIG. 1.

FIG. 3 is a detail of the concentrator in another embodiment.

FIG. 4 is a scheme of apparatus in another embodiment.

### DETAILED DESCRIPTION OF THE INVENTION

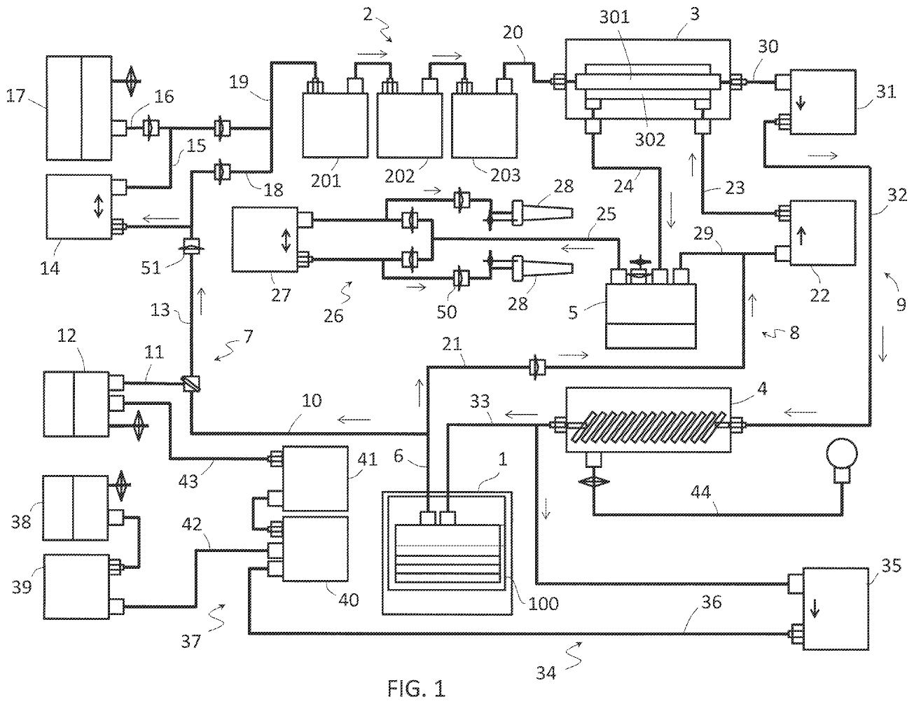

The illustrated apparatus (FIG. 1) which can be called bioreactor essentially comprises: an incubator 1, a thermal conditioner 2, a concentrator 3, a gaseous conditioner 4, an exosome collector 5.

The incubator 1 contains a culture stack 100. A continuous flow of a product 6 which contains exosomes dispersed in an aqueous culture medium is taken from the incubator 1. Said product 6 is essentially an aqueous solution containing exosomes released from the cell culture.

The product 6 is fed to the concentrator 3 through two separate feed lines 7 and 8, which respectively feed two sides of the concentrator 3 communicating with each other via a semi-permeable wall, as will be better described below. A return line 9 for returning a diluted product (medium depleted of exosomes) from the concentrator 3 to the incubator 1 is also provided. The gaseous conditioner 4 is provided along said return line 9.

The arrows in FIG. 1 next to the flow lines indicate the flows during normal operation of the apparatus.

The line 7 feeds a first part 10 of the product 6 to the concentrator 3.

A waste material can be extracted from the product 10 through the line 11, said waste material being then collected in a tank 12. Said line 11 can also be used to extract air during an initial setup step.

The remaining product 13 is fed to the concentrator 3 with a reversible pump 14.

During the direct operation of the pump 14, the product 13 is pumped through the delivery branch 15 to the thermal conditioner 2. The pump 14 is also connected to a fresh medium supply line 16 which allows the medium to be reintegrated in the circuit. The fresh medium is taken from a tank 17 through the pump 14 operating in the opposite direction, using in this case the branch 15 for the suction of the fresh medium from the tank 17 and the branch 18 for the delivery of the fresh medium together with the unidirectional action of the valves on the lines.

The product 19 entering the thermal conditioner 2 therefore results from the first part of product 10, taken from the incubator 1, after a possible extraction of the waste 11 and/or integration with fresh medium 16.

The thermal conditioner 2 comprises a first temperature sensor 201, a heater 202 and a second temperature sensor 203, the two sensors being respectively upstream and downstream of the heater.

The temperature measurement of the two sensors 201 and 203 is used by a control system to regulate the heat input of the heater 202 and, consequently, allows accurately adjust the temperature of the thermally conditioned product 20 at the inlet of the concentrator 3.

It should be noted that the incubator 1 is inserted in a closed and sterile loop (or ring) together with the devices for the concentration of the exosomes starting from the product containing exosomes which is continuously extracted from the incubator itself.

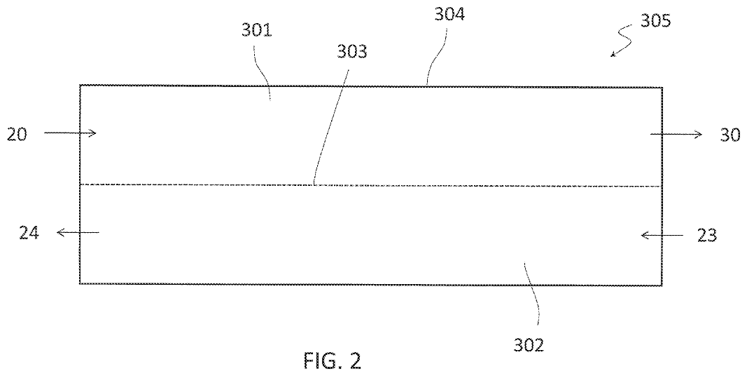

FIG. 2 is a scheme of the concentrator 3 which for example is made as a flat rectangular tangential filter and comprises a first chamber 301, a second chamber 302, in which said two chambers 301 and 302 are communicating with each other via a semi-permeable wall 303. Said wall 303 allows the passage of the aqueous medium but is substantially impermeable to the exosomes. The whole is contained in a vessel or container 304. Advantageously the chambers 301, 302 are part of rectangular cartridges 305.

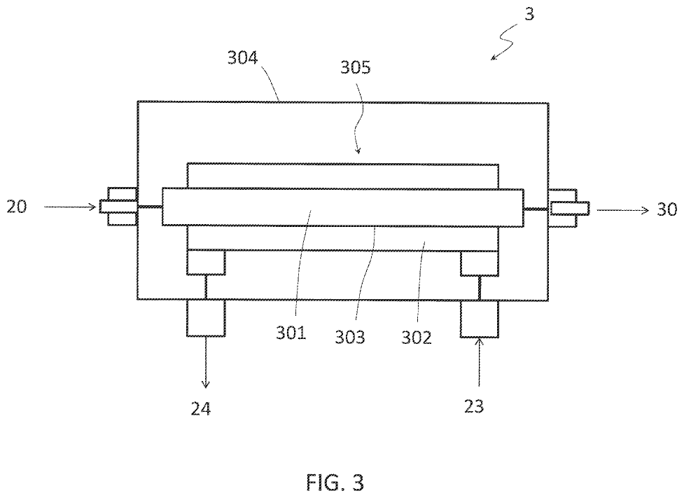

FIG. 3 shows another embodiment of the concentrator 3 in which the chambers 301 and 302 are coaxial. It should be noted that other geometries may be used within the scope of the present invention.

The material 20 exiting the thermal conditioner 2 feeds the first chamber 301 at controlled temperature.

The line 8 feeds a second part 21 of product to the second chamber 302 of the concentrator 3. The product 21 is fed through a pump 22 and a line 23 of delivery of the pump and of entry into said second chamber 302.

It should be noted that the product flows 20 and 23 are advantageously fed in a counter-current manner in the two chambers 301 and 302. In this way, a correct differential is maintained between the concentrations of exosomes in the two product flows along the longitudinal extension of the concentrator 3.

The product 23 entering the chamber 302, fed by the branch 8, is at a higher pressure than the product 20 entering the chamber 301 and fed by the branch 7. Consequently there is a positive pressure differential between the chamber 302 (at higher pressure) and the chamber 301. This pressure differential for example is from 0.1 bar to 2.5 bar. Due to the pressure differential and the semi-permeable wall 303, during the transit of the flows 20 and 23 through the concentrator, the aqueous medium contained in the product 23 tends to pass from the chamber 302 to the chamber 301.

Consequently, the product circulating in the second chamber 302 is enriched with exosomes. An enriched product 24 is extracted from the second chamber 302. Said enriched product 24 is collected in the collector 5. The product 24 contains a particulate rich in exosomes.

Said collector 5 allows extraction of a concentrate 25 of exosomes. A concentrate collection device 26 moved by a pump 27 allows the concentrate 25 to be collected, for example in containers 28. Said pump 27 is reversible so that it can operate in two directions like the previously described pump 14.

The device 26 comprises a series of unidirectional valves 50 so that the pump 27 can alternately extract the concentrate 25 from the collector 5 and pump it into one of the containers 28.

The enriched product 24 is recirculated to the pump 22 via the line 29.

The diluted product 30 exiting the first chamber 301 (diluted due to the passage of the aqueous medium in the concentrator) is recirculated to the incubator 1 through the line 9 and the gaseous conditioner 4. In this way the aqueous medium is continuously recirculated in a closed line.

More specifically, a pump 31 feeds the diluted product 30 to the gaseous conditioner 4, through a line 32.

Said gaseous conditioner 4 is capable of enriching the product with oxygen and/or carbon dioxide. The conditioned product 33 exiting the gaseous conditioner 4 is reintroduced into the incubator 1. The addition of gas to the material 32, with the gaseous conditioner 4, allows controlling the oxygenation and the pH of the incubator 1. The figure shows an O₂ and/or CO₂ feed line 44.

Advantageously, a sampling line 34 is provided which, by means of a pump 35, takes a sample 36 of the product exiting said gaseous conditioner 4 and feeds it to a pH analyser 37.

Said analyser 37 provides a pH value which can be used by the control system to control the administration of oxygen and/or CO₂ with the gaseous conditioner 4, so that a desired pH of the conditioned product 33 recirculated to the incubator 1 is obtained. In particular, the control system defines a necessary quantity of oxygen and/or CO₂ to be fed to the incubator 1 based on the pH reading given by the analyser 37, and consequently controls the enrichment of oxygen and/or CO₂ delivered from the gas conditioner 4.

The pH analyser is advantageously of the non-contact optical type and comprises: reagent tank 38, reagent pump 39, mixer 40, optical analyser 41.

The sample (sampled solution) 36 is mixed with reagent 42 and subjected to analysis; the analysed sample (contaminated with the reagent) is disposed of via the line 43 in the aforementioned tank 12 which also collects the waste 11 extracted from the product flow 10.

It should be noted that the described pH analyser is suitable for clinical grade cultures in which introducing a colorimetric reagent in the culture is not allowed. The reagent 42 in fact only comes into contact with the sample 36 which is subsequently disposed of, but does not enter the main circuit represented by the lines 7, 8 and 9 and related devices.

The apparatus also comprises a number of valves (such as the valves shown in FIG. 1) which allow for the control of the various flows. Said valves are known per se and do not need to be described. For example one of these valves, on the line 13, is indicated in FIG. 1 with the numerical reference 51. Advantageously, the valves are of the unidirectional type and do not require any actuation, being controlled by the flows circulating in the system.

The control system governs the pumps, the selectors and the thermal conditioner, obtaining the desired circulation of the product, extraction of exosomes and in-line integration of fresh medium. The unidirectional valves are controlled indirectly through the flows created by the different pumps in the system.

In variants of the invention other sensors and/or actuators can be provided. For example, some variants of the invention may provide for one or more of the following: temperature sensors; pH sensors; pressure sensors; at least one glucose sensor to quantify the wear of the culture medium; at least one lactic acid sensor to quantify the level of toxic metabolites produced during culture; other implementations (e.g. regulation of gas flow) for further improvements in the process control.

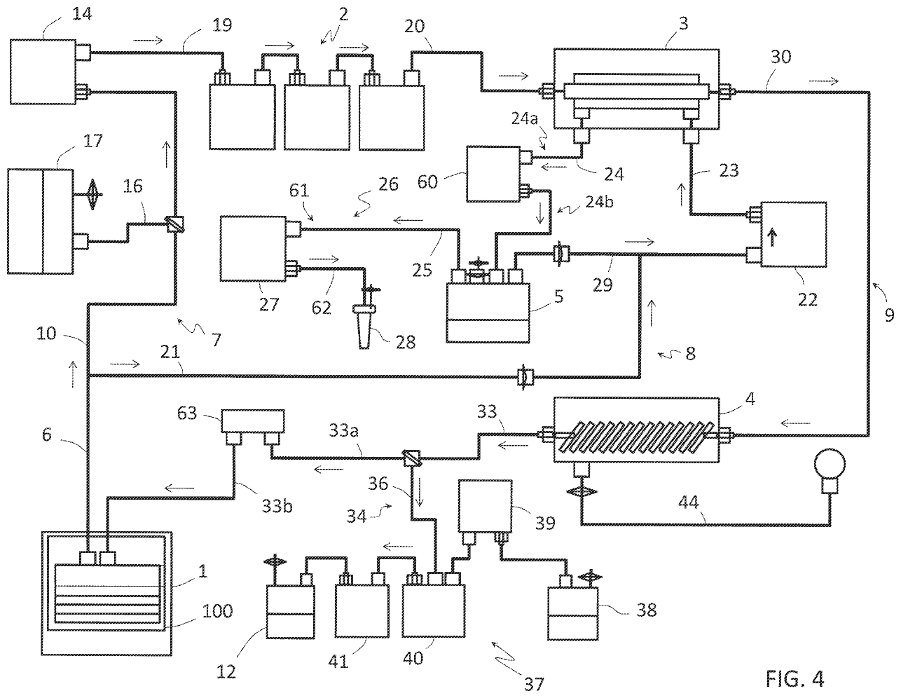

The variant illustrated in FIG. 4 is now described.

In said variant, the essential components of the apparatus are the same as in FIG. 1 and comprise: the incubator 1, the thermal conditioner 2, the concentrator 3, the gaseous conditioner 4, the exosome collector 5. The details which are equivalent to those of FIG. 1 are indicated with the same numerical references. Also in FIG. 4 the arrows indicate the direction of the flows.

In the scheme of FIG. 4 a pump 60 is provided directly on the extraction line of the enriched product 24 from the concentrator 3 to the collector 5. This location of the pump 60 can be preferred so as to ensure a better control of the process and in particular of the transfer of the product 24 from the concentrator 3 to the collector 5.

The extraction line of the enriched product 24 consequently comprises a first branch 24a from the concentrator 3 to the pump 60 and a second delivery branch 24b from the pump 60 to the collector 5.

Through this location of the pump 60 directly on the extraction line, the extraction process is less sensitive to parameters such as the response or pressure drop of the valves, circuits and pumps themselves. Since these parameters are not always precisely known, the positioning of the pump 60 of FIG. 4 allows a more accurate and deterministic process control.

Another feature of the diagram in FIG. 4 is a simplification of the collection device 26 of the concentrate 25. In this simplified variant, the pump 27 is directly connected to the collector 5 and to the container 28. The pump 27 sucks the concentrate 25 directly from the collector 5 via a line 61 and sends it to the container 28 by means of a line 62. This variant does not require the pump 27 to be reversible and requires fewer valves than the analogous circuit of FIG. 1.

Another characteristic of the variant of FIG. 4 is that the sample 36 intended for analysis is taken directly from the delivery line of the conditioned product 33 from the gaseous conditioner 4 to the incubator 1. With this arrangement, the pump 35 of FIG. 1 is not necessary and the device requires one pump less. The remaining product 33a is directed to the incubator 1.

Other components of the analyser system 37 can be similar to the variant of FIG. 1 and comprise for example: reagent tank 38, reagent pump 39, mixer 40, optical analyser 41.

Furthermore, it should be noted that the waste collection tank 12 is advantageously connected to the said analyser system 37. The waste flow is separated only from the sampling line 34. This allows simplifying the delivery line from the incubator 1 to the thermal conditioner 2 and the concentrator 3, in particular by allowing a non-reversible pump 14 to be used. The connection with the tank 17 of the fresh medium 16 is simplified.

The figure shows in particular that the pump 14 is connected directly to the inlet of the thermal conditioner 2. The latter can be made in the manner already described with reference to FIG. 1.

A simplification of the medium return line should also be noted. In particular, the diluted product line 30 connects the exit of the concentrator 3 directly to the gaseous conditioner 4. This simplification is made possible by the different positioning of the pumps and pump 31 in FIG. 1 is no longer needed.

FIG. 4 also shows an optional filter 63 which is installed between the gaseous conditioner 4 and the incubator 1 and which is adapted to remove any air bubbles from the flow 33a. The product 33b exiting the filter 63 is directed to the incubator 1.

The apparatus can be usefully made in the form of a closed kit in which the main circuit between incubator 1, thermal conditioner 2, concentrator 3, gas conditioner 4 and collector 5 is substantially closed and isolated from the user. The user interacts only with the systems for sampling the exosome concentrate and for introducing oxygen and/or CO₂. The use is within reach of unskilled personnel and the same also reduces the risk of contamination.

The invention allows achieve the above mentioned scopes. The apparatus according to the invention operates in a closed and sterile line and in a continuous manner, also allowing the production of large quantities of exosomes.

1. Continuous process for the production of exosomes, comprising:
a) cell culture with release of exosomes carried out in an incubator obtaining from said incubator a product containing exosomes and an aqueous culture medium;
b) continuously feeding a first portion of said product to a first side of a concentrator device;
c) continuously feeding a second portion of said product to a second side of said concentrator device;
d) wherein said first side and said second side of the concentrator device are in communication with each other via a semi-permeable wall which is permeable to the aqueous culture medium and substantially impermeable to the exosomes;
e) wherein the transit of a first flow of said product and a second flow of said product respectively through the first side and the second side of the concentrator device causes a passage of the aqueous culture medium from the second side towards the first side, across said wall, and a consequent increase in the concentration of exosomes in the product which transits through the second side;
f) collecting a product enriched with exosomes from said second side of the concentrator device; and
g) collecting a diluted product from said first side of the concentrator device and recirculating said diluted product to said incubator,
wherein the aforesaid process steps a)-g) are carried out in a closed loop comprising the cell culture in the incubator and the concentration of the exosomes in the concentrator device.

2. Process according to claim 1, further comprising the steps of: sending the product enriched with exosomes, which is extracted from the second side of the concentrator, in a collector device; extracting from the collector a concentrate of exosomes.

3. Process according to claim 2, wherein part of the product enriched with exosomes sent to said collector is recirculated from the collector towards said second side of the concentrator and wherein also the steps of sending the product from the concentrator to the collector and recirculating the product from the collector to the concentrator are carried out in a closed loop.

4. Process according to claim 1, wherein the product is fed into the second side of the concentrator at a higher pressure than the product fed into the first side of the same concentrator.

5. Process according to claim 1, wherein the passage of the culture medium from the second side to the first side of the concentrator is substantially unidirectional.

6. Process according to claim 1, further comprising the addition of fresh culture medium to the product directed to the first side of the concentrator.

7. Process according to claim 1, wherein the product is fed to the first side of the concentrator at a controlled temperature.

8. Process according to claim 7, further comprising a thermal conditioning of the product fed to said first side of the concentrator, before entry of said material into the concentrator.

9. Process according to claim 8, further comprising a measurement of the temperature of the product respectively before and after the thermal conditioning step and wherein said thermal conditioning step is managed on the basis of the temperatures thus measured so as to bring the culture material entering the first side of the concentrator to a desired controlled temperature.

10. Process according to claim 1, further comprising the extraction of a waste flow from said first portion of product, before entry into the concentrator and before the possible addition of fresh medium.

11. Process according to claim 1, further comprising a step of conditioning of diluted product (30) extracted from the first side of the concentrator and directed to a recirculation in the incubator, before the re-introduction of said diluted product into the incubator.

12. Process according to claim 11, wherein the conditioning comprises the addition of oxygen and/or carbon dioxide to the diluted product.

13. Process according to claim 11, further comprising: extracting a sample of product after the conditioning step and before introduction into the incubator; subjecting said sample to a pH measurement preferably by means of colorimetric analysis.

14. Process according to claim 12, wherein the addition of oxygen and/or carbon dioxide during the conditioning step is controlled depending on the value of pH measured in the sample, to keep the value of pH in the incubator within a desired range.

15. Process according to claim 1, wherein the circulation of the product between the incubator and the concentrator as well as the steps of thermal conditioning and conditioning with addition of oxygen and/or carbon dioxide are performed continuously and in a sterile and closed line.

16. Process according to claim 1, wherein the treatment of the product extracted from the incubator does not include any centrifugation step.

17. Apparatus for producing exosomes comprising:
an incubator for preparing, through cell culture with release of exosomes, a product containing exosomes and an aqueous culture medium;
a concentrator adapted for separating exosomes from the aqueous culture medium, wherein said concentrator comprises a first side and a second side which communicate with each other via a semi-permeable wall, said wall being permeable to the culture medium and substantially impermeable to the exosomes;
a first feed line adapted for feeding a part of the product from the incubator to the first side of the concentrator;
a second line adapted for feeding another part of the product from the incubator to a second side of said concentrator device;
a line for collecting product enriched with exosomes from said second side of the concentrator; and
a return line for returning product exiting the first side of the concentrator to the incubator,
wherein said devices are arranged to operate in a closed loop.

18. Apparatus according to claim 17, further comprising an collector of exosomes which is connected to said collection line to receive the product enriched with exosomes collected from the second side of the concentrator.

19. Apparatus according to claim 18, further comprising a pump which is located directly on the extraction line of enriched product from said concentrator towards said collector.

20. Apparatus according to claim 17, further comprising a thermal conditioning device adapted to heat said product, located along said first line upstream of the inlet into the first side of the concentrator, so as to control the temperature of the product entering said first side of the concentrator.

21. Apparatus according to claim 1, further comprising a gas conditioning device adapted to supply oxygen and/or carbon dioxide to product passing through said return line and located along it.

22. Apparatus according to claim 17, wherein the first line comprises a device for extracting waste product and a device for reintegrating fresh medium.

23. Apparatus according to claim 17, wherein the first side and the second side of the concentrator comprise a cartridge preferably having a rectangular shape and/or coaxial cylindrical shape.

24. Apparatus according to claim 17, wherein the incubator, the concentrator, the thermal conditioning device and the gas conditioning device and, if present, the collector, are located in a closed circuit isolated from the external environment.
